(19) United States
(12) Patent Application Publication
(10) Pub. No.: US 2017/0312324 A1
(43) Pub. Date: Nov. 2, 2017

(54) USE OF MAITAKE AND MAITAKE GRIFOLAN D COMPONENT IN PREPARATION OF ANTIDEPRESSANT DRUG

(71) Applicant: Yunnan University, Kunming, Yunnan (CN)

(72) Inventors: Jing DU, Kunming, Yunnan (CN); Chunjie XIAO, Kunming, Yunnan (CN); Hongkun BAO, Kunming, Yunnan (CN); Ming ZHU, Kunming, Yunnan (CN)

(73) Assignee: Yunnan University, Kunming, Yunnan (CN)

(21) Appl. No.: 15/523,307

(22) PCT Filed: Oct. 28, 2015
(86) PCT No.: PCT/CN2015/093087

§ 371 Date: Apr. 28, 2017

(30) Foreign Application Priority Data
Oct. 28, 2014 (CN) 201410588545.9
Jun. 10, 2015 (CN) 201510315301.8

(51) Int. Cl. A61K 36/07; A61K 9/08; A61K 9/00; A61K 31/715

(52) U.S. Cl. CPC A61K 36/07; A61K 31/715; A61K 9/08; A61K 9/0019; A61K 2236/331; A61K 2236/37

(57) ABSTRACT

The present invention “use of Maitake and Maitake polysaccharide D Fraction in manufacture of medicaments for anti-depression” belongs to the field of pharmaceuticals, which characterized in that the Maitake whole herb, Maitake extract or Maitake polysaccharide D fraction is used in the preparation of antidepressants. The medicament can be used to treat or prevent depression, which has a significant antidepressant effect, not only strong effect, but also no conventional antidepressant medicaments toxicity, side effects, such as induced epilepsy, ataxia and other shortcomings. Therefore, they can be used as an excellent antidepressant candidate medicament.

### FIELD

The present invention relates to pharmaceuticals field,especially involving the applications of Maitake and Maitake D polysaccharides fraction in manufacture of medicaments for anti-depression.

### BACKGROUND

Depression has become a common and frequently occurring disease in modern society, whose incidence is still rising. It is featured by emotional distress, waning interest, retardation of thinking, and reduction of words and actions, and patients with major depression may have the ideals of world-weariness or suicide. Such disease will cause pain to patients and their families and lose to the society which are unmatched by many other diseases. According to the report from World Health Organization (WHO), affective disorder has now become the world's fourth-largest disease, and the patients suffering from depression have amounted to 340 million people worldwide. At present, the incidence of depression in China is about 4% and at least 26 million people have depression, causing the total economic burden of RMB 62.2 billion. In Amiracan, it is estimated that 17 million people are suffering from depression and 3 million people are attacked by manic depression, making the cost of treating affective disorder reach as high as USD 44 billion a year (USD 43 billion for coronary). Over the past 40 years of research on affective disorder, its high incidence and mortality are particularly striking. It is reported that suicide rates as high as 10% above and every year 250,000 people commit suicide in China.

Currently, the main medicament used for treating depression is serotonin reuptake inhibitor (SRI) etc., which however has disadvantages such as slow onset, narrow effect spectrum, big side effect, and easy relapse after medicament withdrawal. Now, it is in urgent need of more effective medicaments and treatment methods with fast onset and small side effects in the field of antidepressant drugs.

Current antidepressants and their weaknesses: current antidepressants can be broadly divided into three categories: monoamine oxidase inhibitors, tricyclic antidepressants and heterocyclic antidepressants. The existing antidepressant medicaments are not far from satisfying the needs of patients, which the shortcomings of the main focus on the performance of slow onset, narrow antidepressant spectrum, large side effects, easy to relapse and so on. It is thus indeed imminent to develop new medicaments with fast onset, broad antidepression spectrum and less toxic and side effects, which will bring huge economic returns to the society.

The preparation of the antidepressant medicament according to the present invention relates to the Maitake extract, which is derived from a fungus Maitake (maitake), also known as Grifola frondosa, other names are Polyporus Frondosus, Cloud mushrooms, Thousand-buddha bacteria, chestnut mushrooms, and lotus mushroom. The Latin name is Griflola frondosa (Dick. Fr.) S. F. Gray. Grifola frondosa attaches to the fungi, Basidiomycotina, Hgmenomycetes, Holobasidiomyceidae, Aphyllophrales, Polyporaceae, Grigola, which is a large, edible, medicinal fungus, native to North America, also found in northeastern Japan. Grifola frondosa (Maitake) as a fungal species, the earliest discovered by the Italians, therefore, the Maitake was named after the Italians.

At present, the extract of the Maitake has been prepared into drugs, including the Maitake D-fraction, the English name is maitake d-fraction (or maitake D-fraction). It is protein polysaccharide that extracted from the Maitake, this ingredient can be effective to improve human immunity, to effectively control tumor cell growth, to prevent tumor metastasis and recurrence, to reduce the side effects of chemotherapy. Clinical trials have been conducted in a number of countries for the treatment of cancer.

Maitake contains mainly chain beta-(1-6) combined with chain beta-(1-3) glucan as side chain, and contains mainly chain beta-(1-3) combined with chain beta-(1-6) combined for the activity of side chain glucan. Experiments proved that the glucan significantly inhibit the growth of tumor by activating the immune function. Maitake extract including Maitake polysaccharide which structure as shown in FIG. 1.

Japanese scholars were the first to study on the Maitake extract. Ohno et al. extracted a polysaccharide Grifolan (GRN) with antitumor activity from the Maitake fruiting body, confirming that it has a chain of β-1,3-glucan of C6 per 3 glycosyl groups, as shown in FIG. 1, with a molecular weight of about 500,000. Then Ohno studied the conformation of this polysaccharide and found that it exhibited two conformations in the solid state, namely natural and helical. The extracted polysaccharides were of the natural type, and under the severe conditions, the polysaccharides of the two conformations both had significant antitumor activity.

The researchers in China have also carried out research on the Maitake extract. The fruiting body polysaccharide F-D and mycelia M-D were extracted from the Maitake fruit body and mycelia; F-D and M-D are protein-containing proteoglycans, and presence of β-1, 3 and β-1, 6, glucosidic bond. F-D are composed of fucose, arabinose, xylose, mannose, galactose and glucose. M-D is composed of rhamnose, fucose, arabinose, xylose, mannose, galactose, and glucose.

China is the country, which is the earliest to use the fungus for the treatment of the diseases, the fungus has a high medicinal value. The wild Maitake (Grifola frondosa) was light gray, in northern China grows around persimmon, chestnut trees, and elm roots, so in the north China is also known as chestnut mushroom. Our scientists have been ever found that the Maitake extract from the Maitake can be highly effective as an anti-cancer material. Study shows that its anti-cancer activity is also as high as 35% of the chemotherapy drug mitomycin, as well as 16.5 times than Ganoderma lucidum!

The National Cancer Institute of the United States as early as 1992 confirmed that the extract of the Maitake has the effect of anti-human immunodeficiency virus (HIV). Japan's doctor Namba Hiroshi found that Maitake also have effect on breast cancer, lung cancer, liver cancer, as well as improve the tumor chemotherapy, which brought about various adverse reactions, such as lack of appetite, vomiting, nausea, hair loss and leukopenia and so on. In addition, the study also confirmed that the Maitake extract—Maitake D fraction can help to control blood sugar, to inhibit the accumulation of fat cells, to lower blood pressure, and to enhance the immunity. In 1986, Professor Namba Hiroshi of Kobe University of Medicine published a research report on Maitake extract-D (Maitake-D) in Journal of Japanese Pharmacology. The report pointed out that Maitake polysaccharide has A, B, C, D four types, they all have anti-cancer effect, but the D component is of the best effect. The anti-tumor efficacy of the Maitake polysaccharide used alone was 86.6% (1.0 mg/kg body weight). Comparison experiment with the international general use of chemotherapy drugs mitomycin efficacy, the anti-tumor rate of the Maitake extract used alone was 80%, mitomycin alone administration was 45%, while the two combined administration, tumor inhibition rate was 98%. The method, in the United States, Canada, Japan, has been the preferred method of clinical anticancer, known as the “fourth cancer treatment”.

Study on the immune Characteristics of Polysaccharides via America and Japan. Including many clinical experimental studies have found that the Maitake extract can effectively activate the body's immune system, activation of immune cells, such as NK cells, anti-virus T cells, phagocytic cells, activated phagocytic cells, natural killer cells and other immune cells, induced interleukin, Interferon-y, tumor necrosis factor-a and other cytokines secreting. They have synergy with immunotherapeutic drugs (interferon-2β), which can achieve the inhibition of tumor cell growth, induction of cancer cell apoptosis effect.

Today, in more than 20 countries in the world including the United States, Japan, Canada and China, more than 3,000 cancer doctors have applied and validated the clinical efficacy of the Maitake extract, which has also been used as a tumor treatment of the first line of clinical medication in Europe and the United States and other western countries. However, after a wide range of literature search, the use of Maitake or Maitake polysaccharide D fraction as a medicinal component of the treatment of depression has not yet been reported.

### SUMMARY

The present invention provides the applications of Maitake whole herb or the Maitake Polysaccharides D fraction as an antidepressant medicament and the antidepressant medicament prepared by Maitake whole herb or Maitake Polysaccharides D fraction.

This medicament has the advantages such as remarkable antidepressant effects and small side effects and so on. The technical solutions are as follows:

An application of Maitake in the preparation of antidepressant medicaments is characterized as that the medicinal component of the antidepressant is selected from group of consisting of Maitake whole herb, Maitake extract, Maitake Polysaccharides D fraction, or Maitake Polysaccharides D fraction salts, esters or the glycosides synthesized by Maitake Polysaccharides D fraction and polysaccharides.

Said Maitake extract refers to product from following process: marinating the powder of the Maitake fruit body in pure hot water of 98° C. for 3 hours, the quality ratio of material to water is 1: 30, pH 6.5-7.0, and then concentrating the solution.

The mentioned antidepressant medicament refers to a medicament that used for preventing or treating depression.

The mentioned medicament is in dosage form of table, capsule, powder, solution, suspension, injecta or drip.

A kind of antidepressant medicament, it is characterized in: the medicinal component is selected from group of consisting of Maitake whole herb, Maitake extract, Maitake Polysaccharides D fraction, Maitake Polysaccharides D fraction salts, esters or the glycosides synthesized by Maitake Polysaccharides D fraction and polysaccharides.

The mentioned Maitake extract refers to product from following process: marinating the powder of the Maitake fruit body in pure hot water of 98° C. for 3 hours, the quality ratio of material to water is 1: 30, pH 6.5-7.0, and then concentrating the solution.

The mentioned antidepressant refers to preventing or treating depression.

Said medicament is in dosage forms of table, capsule, solution, suspension, injecta or drip.

The mentioned medicament also contains pharmaceutically acceptable adjuvant.

The mentioned medicament also contains pharmaceutical ingredient that plays a positive role in treating depression together with Maitake whole herb, Maitake extract, or Maitake Polysaccharides D fraction, or application a component that enhances the stability of Maitake whole herb, Maitake extract, or Maitake Polysaccharides D fraction.

An antidepressant method characterized in that the treatment comprises providing an effective doses of medicament to subject on time, wherein medicinal component of mentioned medicament is selected from group of consisting of the Maitake whole herb, Maitake extract, or Maitake Polysaccharides D fraction.

The mentioned Maitake extract refers to product from following process : marinating the powder of the Maitake fruit body in pure hot water of 98° C. for 3 hours, the quality ratio of material to water is 1: 30, pH 6.5-7.0, and then concentrating the solution.

The mentioned providing an effective doses of medicament to subject on time refers to providing Maitake Polysaccharides D fraction or Maitake extract with a dose of from 0.5 mg-1000 mg/kg of body weight per day, or providing the Maitake whole herb together with food by a quality ratio of 1: 1-4 between the Maitake whole herb and food.

The mentioned providing effective doses of medicament to subject on time refers to providing Maitake Polysaccharides D fraction or Maitake extract by 5-15 mg/kg body weight per day.

The method of administration is oral, infusion or injection; the subject is a mammal, the mentioned mammal comprising human.

The inventors of the present invention have found that the Maitake whole herb, or Maitake Polysaccharides D fraction has a significant and strong antidepressant effect on the mammal. Experiments showed that in mammalian mice experiment, the Maitake whole herb, or Maitake Polysaccharides D fraction demonstrated obvious and strong antidepressant effect, and antidepressant effect can continue to last. Depression patients generally complained of inflammatory pain symptoms, such as headache, stomach pain, back pain, etc., Maitake whole herb, or Maitake Polysaccharides D fraction at the same time of antidepressant, but also can improve the patient's inflammatory symptoms. Many patients with tumor disease after treatment with chemotherapy, radiotherapy, concurrent depression, Maitake whole herb, or Maitake Polysaccharides D fraction can also play a role in both anti-tumor and antidepressant effect. Therefore, Maitake extract can comprehensively improve the various symptoms of depressive patients, including emotional symptoms of depression, pain, inflammation and so on. Its antidepressant effect is characterized by significant, rapid, and long-lasting in animal models. The currently used antidepressants has a latency to be effective, generally takes several weeks, and the patient heavily depends on these medicaments. The antidepressant medicament provided by the present invention is fast acting and has persistent action for several days after being stopped.

The application of Maitake whole herb, or Maitake Polysaccharides D fraction of the present invention is commercially available. The Maitake Polysaccharides D fraction is a Maitake D-fraction with an average molecular weight of 1 million, and its structure as shown in FIG. 1. It is a kind of polysaccharide compound isolated from the fungal polyporales, Griflola frondosa genus, Griflola frondosa. It is safe and can be taken by humans for a long time and has no known side effects so far. There is no epilepsy, ataxia and other shortcomings as that of conventional antidepressant medicament observed in the course of experiment. It has the potential to become a new antidepressant of fast-acting, efficient, non-toxic side effects, and has obvious industrial utility and great commercial value.

The medicament screening tests adopted in this invention is: Mice forced swimming and tail suspension tests. They are two kinds of more commonly used animal behavior despairing depression model tests and can ensure the reliability of screening results.

Mice forced swimming test has been used for the screening test of many antidepressant medicaments. And most of antidepressant medicaments with clinical therapeutic effects have been proved to be able to effectively reduce the immobility time in the forced swimming test. “Immobility” herein refers to that “animals stop struggling in the water, or stay in a floating state, only showing nostrils for breathing with only small body movement to keep the head floating on the water”. Before the test, the medicaments to be screened are given. The animals in the condition of forced swimming cannot escape from the harsh environment, leading to despairing animal behaviors. This model method is simple and reliable and it has been widely used in the screening and evaluation of antidepressant medicaments.

In the mice tail suspension test, mice present the special quiet immobility state without any struggle in the tail suspension state, and antidepressant medicaments can obviously shorten the duration of the immobility state. When tested, mice tails are fixed with clips, hanging upside-down. Do not make the mouse tail twist or fold. The time of immobility is recorded. The immobility indicator is “animal has no body movement in addition to breathing”. Tail suspension test is very sensitive to a variety of antidepressant medicaments, and the interference of temperature and animal movement dysfunction in the swimming test are avoided, so using some rat species to screen antidepressant medicaments can effectively verify and supplement the results of forced swimming test.

The application of Maitake extract in resisting depression provided in this present invention is not limited to that of the Maitake whole herb with the structural formula in FIG. 1 (Maitake Polysaccharides D fraction). It is a conventional experiment skill for technicians in this field to prepare Maitake Polysaccharides D fraction salts and esters by Maitake Polysaccharides D fraction or prepare glycosides synthesized by Maitake Polysaccharides D fraction and sugar. Maitake Polysaccharides D fraction salts and esters or prepare glycosides synthesized by Maitake Polysaccharides D fraction and sugar with Maitake Polysaccharides D fraction has a similar pharmacological effect that can be reasonably anticipated by technicians in this field. Therefore, this present invention also contains application of Maitake Polysaccharides D fraction salts, esters or the glycosides synthesized by Maitake Polysaccharides D fraction and sugar in resisting depression.

The application of Maitake whole herb or Maitake Polysaccharides D fraction resisting depression provided in this present invention indicates that, the adjuvant that will not affect to medicament efficacy, such as carrier and excipient, can be added into the prepared antidepressant medicament based on knowledge of the technician in this field. Since this medicament can be taken effectively by oral, injection and subcutaneous embedding, etc., the dosage form can be varied.

The antidepressant medicament provided in this invention can also include other ingredients that are used cooperatively with Maitake whole herb / Maitake Polysaccharides D fraction or its derivatives, as well as pharmaceutical ingredients that have positive effects on treatment of depression.

The technicians in this field can add a stabilizing agent that increases the stability of the Maitake Polysaccharides D fraction. As long as the obtained antidepressant medicament does not affect the efficacy of Maitake Polysaccharides D fraction, it is in the scope of protection this invention.

The antidepressant preparation provided in this invention includes two aspects, namely prevention and treatment.

Under permit of Patent Law, this present invention also requires protecting the application of Maitake whole herb or Maitake Polysaccharides D fraction in resisting and treating depression; in the animal model test, the dose range of stabilizing the antidepressant effect was daily injected the low dose (5 mg/kg), the medium dose (8 mg/kg) and the high dose (12.5 mg/kg) of the Maitake Polysaccharides D fraction. Or in accordance with the daily intake of Grifola frondosa and food ratio of 1:4, 1:2 or 1:1 provides Grifola frondosa.

Since the shortening of the immobility time of the classical depression animal model may be due to the effect of the central excitability of the medicament, in order to determine whether the Maitake whole herb or Maitake Polysaccharides D fraction will increase the activity of the animal, this invention was also performed the Open field test to examine the autonomic activity of the mice, and to avoid interference with the central stimulant. The results showed that the each dose of Maitake whole herb or Maitake Polysaccharides D fraction had no significant effect on the autonomic activity of mice, so the possibility of false positive results could be ruled out, which proved that the antidepressant effect of the medicament was reliable.

### BRIEF DESCRIPTION OF THE DRAWINGS

FIG. 1. The chemical structural formula of Maitake Polysaccharides D fraction.

FIG. 2. Treatment with Maitake Polysaccharides D fraction to mice after 60 minutes—data in tail suspension test (TST) :

Vertical coordinate: tail suspension immobility (%control);

Horizontal coordinate: from left to right: Maitake Polysaccharides D fraction control (0.9% saline), Maitake Polysaccharides D fraction low dose (5 mg/kg), Maitake Polysaccharides D fraction medium dose (8 mg/kg), Maitake Polysaccharides D fraction high dose (12.5 mg/kg) , Imipramine control (15 mg/kg).

FIG. 3. Treatment with Maitake Polysaccharides D fraction to mice after 60 minutes—data in forced swimming test (FST):

Vertical coordinate: forced swimming immobility (%control);

Horizontal coordinate: from left to right: Maitake Polysaccharides D fraction control (0.9% saline), Maitake Polysaccharides D fraction low dose (5 mg/kg), Maitake Polysaccharides D fraction medium dose (8 mg/kg), Maitake Polysaccharides D fraction high dose (12.5 mg/kg) , Imipramine control (15 mg/kg).

FIG. 4. Treatment with Maitake Polysaccharides D fraction to mice after 5 days—data in tail suspension test (TST):

Vertical coordinate: tail suspension immobility (%control);

Horizontal coordinate:from left to right: Maitake Polysaccharides D fraction control (0.9% saline), Maitake Polysaccharides D fraction low dose (5 mg/kg), Maitake Polysaccharides D fraction medium dose (8 mg/kg), Maitake Polysaccharides D fraction high dose (12.5 mg/kg) , Imipramine control (15 mg/kg).

FIG. 5. Treatment with Maitake Polysaccharides D fraction to mice after 5 days—data in forced swimming test (FST):

Vertical coordinate: forced swimming immobility (%control);

Horizontal coordinate: from left to right: Maitake Polysaccharides D fraction control (0.9% saline), Maitake Polysaccharides D fraction low dose (5 mg/kg), Maitake Polysaccharides D fraction medium dose (8 mg/kg), Maitake Polysaccharides D fraction high dose (12.5 mg/kg) , Imipramine control (15 mg/kg).

FIG. 6. Treatment with Maitake Polysaccharides D fraction to mice after 5 days—data in Open field test (FST):

Vertical coordinate: the center area distance (% meter);

Horizontal coordinate: from left to right: Maitake Polysaccharides D fraction control (0.9% saline), Maitake Polysaccharides D fraction low dose (5 mg/kg), Maitake Polysaccharides D fraction medium dose (8 mg/kg), Maitake Polysaccharides D fraction high dose (12.5 mg/kg) , Imipramine control (15 mg/kg).

FIG. 7. Treatment with Maitake Polysaccharides D fraction to mice after 5 days—data in Open field test (FST):

Vertical coordinate: the total area distance (%meter);

Horizontal coordinate: from left to right: Maitake Polysaccharides D fraction control (0.9% saline), Maitake Polysaccharides D fraction low dose (5 mg/kg), Maitake Polysaccharides D fraction medium dose (8 mg/kg), Maitake Polysaccharides D fraction high dose (12.5 mg/kg) , Imipramine control (15 mg/kg).

FIG. 8. Body weight of the mice after 5 days of treatment with Maitake Polysaccharides D fraction.

FIG. 9. The photo of Grifola frondosa.

FIG. 10. After one day Grifola frondosa treatment to mice—data in tail suspension test:

Vertical coordinate: tail suspension immobility (% of control);

Horizontal coordinate: from left to right: Negative control (0.9% saline), Imipramine positive control (15 mg/kg), Grifola frondosa (1:4), Grifola frondosa (1:2), Grifola frondosa (1:1).

FIG. 11. After one day Grifola frondosa treatment to mice—data in forced swimming test:

Vertical coordinate: forced swimming immobility (% of control);

Horizontal coordinate: from left to right: Negative control (0.9% saline), Imipramine positive control (15 mg/kg), Grifola frondosa (1:4), Grifola frondosa (1:2), Grifola frondosa (1:1).

FIG. 12. The treatment of Grifola frondosa to mice after three days—data in Open field test (FST):

Vertical coordinate: the distance traveled in the total area (% meter);

Horizontal coordinate: from left to right: Negative control (0.9% saline), Grifola frondosa (1:4), Grifola frondosa (1:2), Grifola frondosa (1:1).

FIG. 13. The treatment with Grifola frondosa to mice for three days—data in Open field test (FST):

Vertical coordinate: the distance traveled in the center area (% meter);

Horizontal coordinate: from left to right: Negative control (0.9% saline), Grifola frondosa (1:4), Grifola frondosa (1:2), Grifola frondosa (1:1).

FIG. 14. Treatment with Maitake extract after 3 days to mice—data in tail suspension test (TST):

From left to right: Negative control (0.9% saline), Maitake extract low dose (5 mg/kg), Maitake extract medium dose (8 mg/kg), Maitake extract high dose (12.5 mg/kg) , Imipramine control (15 mg/kg).

FIG. 15. Treatment with Maitake extract after 3 days to mice—data in forced swimming test (FST):

From left to right: Negative control (0.9% saline), Maitake extract low dose (5 mg/kg), Maitake extract medium dose (8 mg/kg), Maitake extract high dose (12.5 mg/kg) , Imipramine control (15 mg/kg).

FIG. 16. Treatment with Maitake extract to mice after 4 days—data in Open field test (OFT) to determine the distance traveled:

From left to right: Negative control (0.9% saline), Maitake extract low dose (5 mg/kg), Maitake extract medium dose (8 mg/kg), Maitake extract high dose (12.5 mg/kg) , Imipramine control (15 mg/kg).

FIG. 17. Treatment with Maitake extract to mice after 4 days in Open field test (OFT) to determine the distance traveled in the center area:

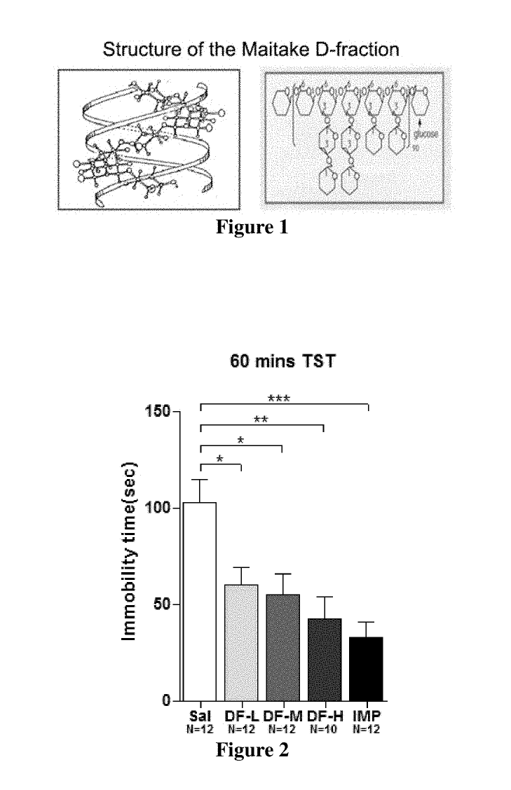

From left to right: Negative control (0.9% saline), Maitake extract low dose (5 mg/kg), Maitake extract medium dose (8 mg/kg), Maitake extract high dose (12.5 mg/kg), Imipramine control (15 mg/kg).

### DETAILED DESCRIPTION

The following implementation plan are only intended to illustrate and demonstrate the discovery of the new use of the Maitake extract of the invention that found in resisting depression, without limiting this invention.

### EXAMPLE 1

### Antidepressant Experiments of Maitake Polysaccharides D Fraction

Experimental materials and methods:

### I. Experimental Details:

1. Animal species: mouse, CD-1 (ICR)

2. Level: SPF level

3. Animal specifications: purchase of initial age and weight (5 weeks old: 20-22 g)

Formal laboratory weeks and body weight (7 weeks old: 30-39 g)

4. Gender: Male

5. Quality inspection unit: Beijing Vital River Laboratories

6. Use: scientific research

7. Sale of the unit: Beijing Vital River Laboratories

8. License number: SCXK (jing) 2012-0001

### II. Experimental Conditions

1. Experimental animals were received on Jul. 15, 2014 from the Beijing Capital Airport on early flight (8:10 take off), at 13:30 pm animals arrived Kunming Changshui Airport cargo center. We chartered a car from Yunnan University to Kunming Changshui Airport cargo center to take experimental animals back to the Yunnan University biological pavilion of East Building, Room 301, animal housing room. Then, let the mice stay in the original box to rest for 2 hours after sub-cage feeding.

2. Each cage housed 4 mice, drinking water and feeding free.

3. Drinking distilled water (laboratory equipment homemade), and mouse food was purchased from Animal Experimental Department of the Kunming Medical University.

4. Twelve hours Light / dark cycle: at 9:00 a.m. lights were on, and at 21:00 p.m. lights were off .

5. Room temperature was at 23° C.

6. The bedding of the cage was replaced every two days. Let the mice have a comfortable living environment.

7. Mice adapted the environment for one week in the new cage before the formal experiment.

### III. Maitake Polysaccharides D Fraction

Maitake D-fraction was prepared from alcohol precipitation from the Maitake drops (Milk Healthy Milk Company), and this method removes any impurities such as glycerin and water, and proceeds as follows:

1) Prepared and marked ten 1.5-ml centrifuge tube, and then weighed empty tube for product calculation;

2) For each 1.5-ml centrifuge tube, we took 200 ul of Maitake drops, added 600 ul 95% ethanol, and then set at room temperature for 2 hours. The precipitation at the bottom of 1.5 ml centrifuge tube was clearly observed.

3) The tubes were centrifuged with high-speed refrigerated centrifuge at 12000 rpm, at 4° C. for 1 hour. At the bottom of the centrifuge tube, we could clearly observe the precipitation. The supernatant was removed into another tube.

4) The 1.5 ml centrifuge tubes with the precipitation were placed in a 37° C. incubator for one hour.

5) We took the 1.5 ml centrifuge tube from the incubator, air dried at room temperature, then determine the weight of the Maitake D-fraction by weight loss method.

6) Approximately 10 mg of Maitake D-fraction was obtained from each 1.5 ml centrifuge tube.

Reference method: 3 times the volume of 95% ethanol alcohol precipitation, wash with acetone, ether, respectively, freeze-dried with a vacuum freeze dryer to get Maitake D-fraction.

### IV. Preparation of the Different Concentrations of Maitake D-Fraction Required Concentration of the Allocation Method for the Mouse Injection:

1. A total of 108.4 mg Maitake D-fraction was obtained from 10 centrifuge tubes by weight loss method, and then 200 ul of 0.9% saline (purchased from the hospital) was added to each 1.5-ml centrifuge tube to dissolve the precipitation at the bottom of the centrifuge tube, and then transfer the thoroughly dissolved solution from the ten 1.5-ml centrifuge tubes to a sterile 7 ml centrifuge tube with a 200 ul pipetting, then add 2 ml 0.9% saline diluted (with a total of 4 ml 0.9% saline).

The final concentration of the Maitake D-fraction solution was 108.4mg/4m1=27.1 mg/ml.

2. The different dose dilutions of Maitake D-fraction were as follows:

| Concentration of | Dilution | Diluted | Medicament | The final dose |
|---|---|---|---|---|
| stock solution | factor | concentration | volume | required |
| 27.1 mg/ml | 1:54.2 | 0.5 mg/ml | 300 ul/30 g | 5 mg/kg |
| 27.1 mg/ml | 1:33.9 | 0.8 mg/ml | 300 ul/30 g | 8 mg/kg |
| 27.1 mg/ml | 1:21.7 | 1.25 mg/ml | 300 ul/30 g | 12.5 mg/kg |

Aliquots of Maitake D-fraction was prepared into 1.5 ml centrifuge tubes according to the dilution factor above, wrapped in the entire 1.5 ml of the centrifuge tube with aluminum foil, sealed, and placed in -80° C. freezer. The aliquots were taken out before use, and then diluted in 0.9% saline according to the calculation of dilution ratio, mixed well before use.

3. The dose dilution factor required for imipramine is as follows:

| Concentration of | Dilution | Diluted | Medicament | The final dose |
|---|---|---|---|---|
| stock solution | factor | concentration | volume | required |
| 15 mg/ml | 1:10 | 1.5 mg/ml | 300 ul/30 g | 15 mg/kg |

Thirty miligram imipramine was weighed, placed in sterile 7m1 centrifuge tube, and then 2ml 0.9% saline dissolution was added (the final concentration of imipramine solution was 30 mg/2 ml=15 mg/ml) to the tube to make the solution. The imipramine solution was aliqoted, sealed, and freezed down in −80° C. freezer. Just before use, the imipramine solution was taken out, and diluted in 0.9% saline according to the calculation of dilution ratio. The solution was mixed well before use.

### V. Behavioral Experiment:

1. The mice had adapted in our new breeding environment for a week before the experiment (the mice we purchased were 5 weeks old, so the mice in our new breeding environment adapt to two weeks before the formal experiment);

2. To start the experiment on Jul. 30, 2014, we prepared the medicine and the equipment needed for the experiment before 10:00 am. Since then the mice were weighed and injected every day at 10:00 am;

3. Experimental arrangements:

| Day 1 | Day 2 | Day 3 | Day 4 | Day 5 | Day 6 |
|---|---|---|---|---|---|
| After 60 | Mice | Mice | After 60 | At the same | At the same |
| minutes of | rest | rest | minutes | time every | time every |
| injection, the | at the | at the | of | day After | day After |
| suspension | same | same | injection, | 60 minutes | 60 minutes |
| test or forced | time | time | the open | of injection, | of |
| swimming | after the | after the | field test | the | injection, |
| test was | day of | day of | was | suspension | anatomical |
| performed | injection | injection | performed | test or forced | experiments |
| | | | | swimming | were |
| | | | | test was | performed |
| | | | | performed | in mice |

4. Forced swimming conditions: room temperature: 23° C., water temperature: 24° C., humidity: 62-67%, replaced the water after two groups of experiments.

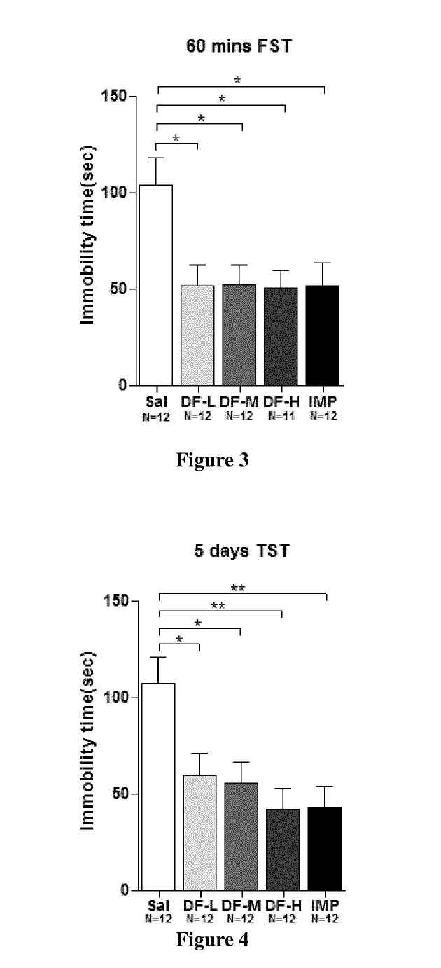

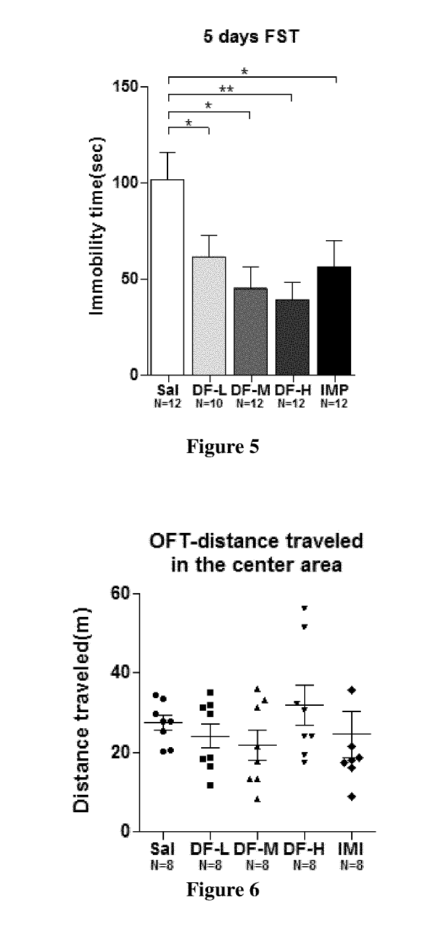

5. Experimental results:
Behavioural experiment results of CD-1 mice were treated with Maitake D-fraction of different doses:
1) The results of the tail suspension test from mice treated with Maitake D-fraction (10-12 repeat), after 60 minutes or 5 days of treatment, were shown in FIG. 2 and FIG. 4, respectively.
2) The results of the forced swimming test from mice treated with Maitake D-fraction (10-12 repeat), after 60 minutes or 5 days of treatment, were shown in FIG. 3 and FIG. 5, respectively.

### Analysis of Experimental Results:

The tail suspension test results of histogram from Maitake D-fraction (10-12 repeat) treated mice after 60 minutes or 5 days were presented in FIG. 2 and FIG. 4. It could be seen that Maitake D-fraction of low dose (5 mg/kg) showed significant antidepressant effect (after 60 minutes: P=0.0099, P<0.01; 5 days: P=0.0151, P<0.05), the medium dose of Maitake D-fraction (8 mg/kg) showed significant antidepressant effect (after 60 minutes: P=0.0068, P<0.001; 5 days: P=0.0078, P<0.01), and the high dose of Maitake D-fraction (12.5 mg/kg) showed significant antidepressant effect (after 60 minutes: P=0.0018, P<0.001; 5 days: P=0.0014, P<0.01). (As shown in FIG. 2 and FIG. 4)

The forced swimming test results of histogram from Maitake D-fraction (10-12 repeat) treatment after 60 minutes or 5 days were presented in FIG. 3 and FIG. 5. It could be seen that Maitake D-fraction of low dose (5 mg/kg) showed significant antidepressant effect (after 60 minutes: P=0.0095, P<0.01; 5 days: P=0.0431, P<0.05), the medium dose of Maitake D-fraction (8 mg/kg) showed significant antidepressant effect (after 60 minutes: P=0.0080, P<0.01; 5 days: P=0.0046, P<0.01), and the high dose of Maitake D-fraction (12.5 mg/kg) showed significant antidepressant effect (after 60 minutes: P=0.0062, P<0.001; 5 days: P=0.0012, P<0.01). (As shown in FIG. 3 and FIG. 5)

3) The results of distance traveled in the center area in open field test after 3 days of Maitake D-fraction (8 repeat) treatment were shown in FIG. 6.

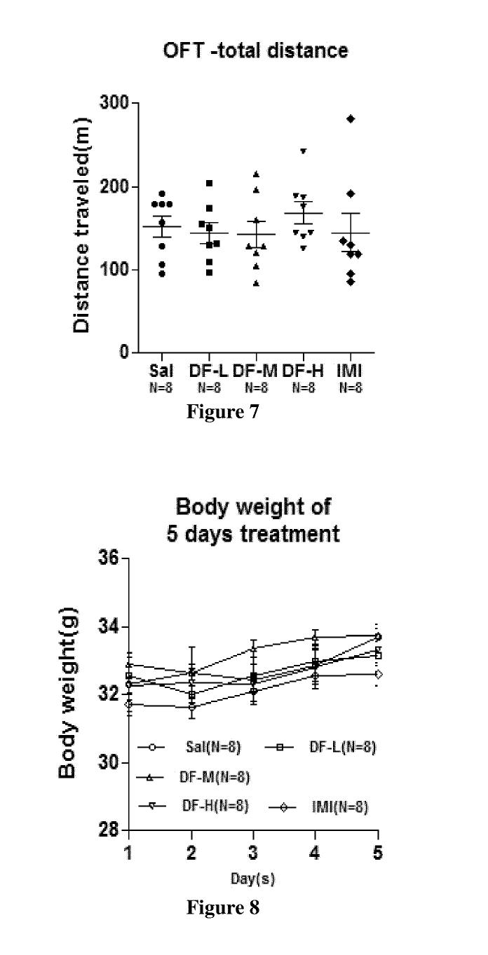

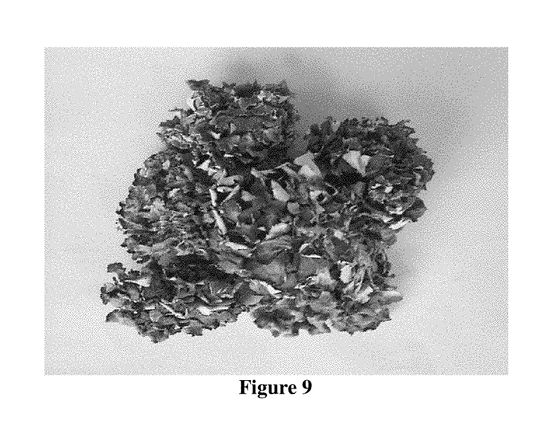

4) The results of distance traveled in the total area in open field test after 3 days of Maitake D-fraction (8 repeat) treatment were shown in FIG. 7.

### Analysis of Experimental Results:

The results of histogram of distance traveled in the center area in open field test after 3 days of Maitake D-fraction (8 repeat) treatment was presented in FIG. 6. It can be seen that Maitake D-fraction of low dose (5 mg/kg), medium dose (8 mg/kg), high dose (12.5 mg/kg) showed no significant effect (P>0.05), and the results further implied that the shortening of the immobilized time of the mice is not caused by the central excitatory effect of the medicament (Maitake D-fraction). (As shown in FIG. 6)

The results of histogram of distance traveled in the total area in open field test after 3 days of Maitake D-fraction (8 repeat) treatment were demonstrated in FIG. 6. It can be seen that Maitake D-fraction of low dose (5 mg/kg), medium dose (8 mg/kg), high dose (12.5 mg/kg) showed no significant effect (P >0.05), and the results further implied that the shortening of the immobilized time of the mice is not caused by the central excitatory effect of the medicament (Maitake D-fraction). (As shown in FIG. 7)

Body weights of the mice after 5 days of treatment were shown in FIG. 8.

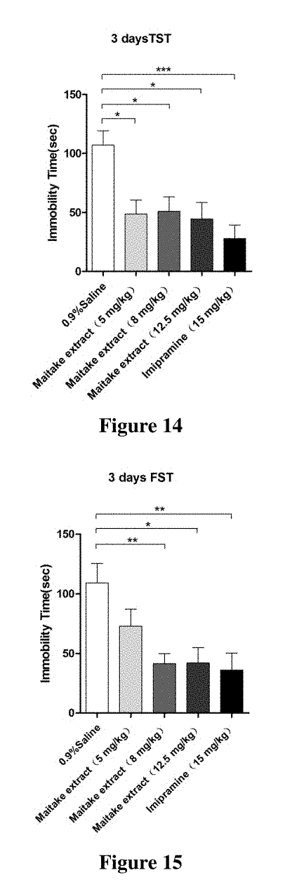

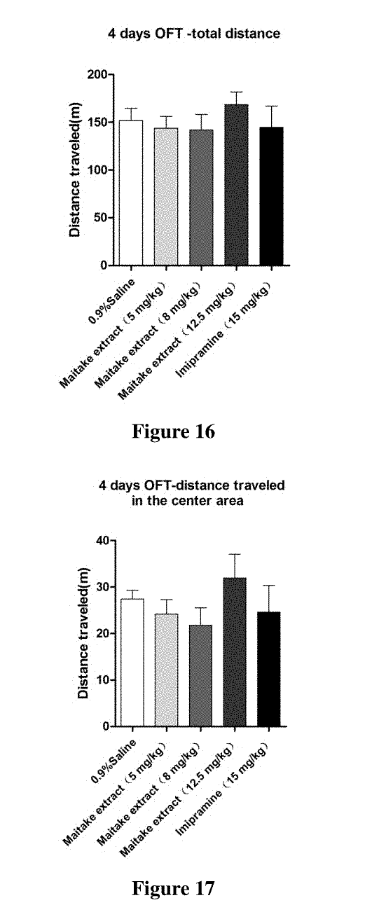

In other examples of the present invention, a directly extracted Maitake extract is used. That means the fine powder of the Maitake fruiting body was extracted with pure hot water (material to water ratio of 1:30). The optimal technological conditions were as follows: pH 6.5-7.0, extracted at 98° C. for 3 hours after the concentration derived. In the mouse experiment, almost exactly the same results as those shown in FIG. 4-7 were obtained, please see FIG. 14-17.

### EXAMPLE 2

### Antidepresaant Experiment of Grifola frondosa Whole Herb (Maitake Whole Herb)

A. Experimental Materials and Methods:

(A) Experimental animal details

1. Animal species: mouse, CD-1 (ICR)

2. Level: SPF level

3. Animal specifications: the initial age and weight purchased (6 weeks old: 22-25 g); Formal laboratory weeks and body weight (7 weeks old: 30-37 g)

4. Gender: Male

5. Quality inspection unit: Beijing Vital River Laboratories

6. Use: scientific research

7. Sale of the unit: Beijing Vital River Laboratories

8. License number: SCXK (jing) 2012-0001

(B) . Experimental Animal Feeding Conditions:

1. Experimental animals arrived on Apr. 21, 2015 from the Beijing Capital Airport early flight (7:40 take off). At 13:30 pm, animals arrived Kunming Changshui Airport cargo center. We chartered a car from Yunnan University to Kunming Changshui Airport cargo center to take experimental animals back to the Yunnan University biological pavilion of East Building, Room 301, animal housing room. Then, let the mice stay in the original box to rest for 2 hours after sub-cage feeding.

2. Each cage housed 4 mice, drinking water and feeding free.

3. Drinking distilled water (laboratory equipment homemade), and mouse food was purchased from Animal Experimental Department of the Kunming Medical University.

4. Twelve hours Light/dark cycle : at 9:00 a.m. lights were on, and at 21:00 p.m. lights were off .

5. Room temperature was at 23° C.

6. The bedding of the cage was replaced every two days.

7. Mice adapted the environment for one week in the new cage before the formal experiment.

(C) Grifola frondosa Details:

1. Grifola frondosa: Purchased from Yunnan wild mushroom Industry Co., Ltd. Vegetatively: Fujian Province

(D) Grifola frondosa and Regular Food with Different Ratio of the Production of Grifola Frondosa-Containing Food.

1. The dry Grifola frondosa were placed in a sterilization pot. Then we set constant temperature at 100° C. and steamed the dry Grifola frondosa for 30 minutes.

2. Then we removed the steamed Grifola frondosa, placed on a clean gauze, dried in the natural sunlight, until dry.

3. The dried Grifola frondosa was then crushed into the powder with a universal crusher and packed in a clean plastic bag.

4. In order to prepare the Grifola frondosa-containing food, we went to mice food production room, in the Experimental Animal Department, Kunming Medical University. The production of different ratio of Grifola frondosa-containing food was performed, according to the weight ratio. Details are as follows:

| | |
|---|---|
| Grifola frondosa dry powder:normal mice food base material | 1:4 |
| Grifola frondosa dry powder:normal mice food base material | 1:2 |
| Grifola frondosa dry powder:normal mice food base material | 1:1 |

(E) The Different Experimental Treatment Groups Were Set Up As Follows, Each Group Had 15 Mice:

Negative control group: eating normal mice food, daily intraperitoneal injection of 0.9% saline according to weight;

Positive control group: eating normal mice food, daily intraperitoneal injection of imipramine (15mg/kg) according to weight;

Grifola frondosa mice food: eating 1:4 Grifola frondosa-containing food, daily intraperitoneal injection of 0.9% saline according to weight;

Grifola frondosa mice food: eating 1:2 Grifola frondosa-containing food,daily intraperitoneal injection of 0.9% saline according to weight;

Grifola frondosa mice food: eating 1:1 Grifola frondosa-containing food, daily intraperitoneal injection of 0.9% saline according to weight;

In order to ensure the consistency of the experiment, due to the establishment of a positive control for the intraperitoneal injection of imipramine, so the other groups were corresponding daily intraperitoneal injection of 0.9% saline according to weight.

(F) The Positive Control Medicament Imipramine for Mice Injection was Prepared as Follows:

| Concentration of | Dilution | Diluted | Medicament | The final dose |
|---|---|---|---|---|
| stock solution | factor | concentration | volume | required |
| 15 mg/ml | 1:10 | 1.5 mg/ml | 300 ul/30 g | 15 mg/kg |

Sixty miligram imipramine was weighed, placed in sterile 7 ml centrifuge tube, and then 4 ml 0.9% saline dissolution was added (the final concentration of imipramine solution was 60 mg/4 ml=15 mg/ml) to the tube to make the solution. The imipramine solution was aliqoted, sealed, and freezed down in −80° C. freezer. Just before use, the imipramine solution was taken out, and diluted in 0.9% saline according to the calculation of dilution ratio. The solution was mixed well before use.

(G) Behavioral Experiment:

1. The mice had adapted in our new breeding environment for a week before the experiment;

2. To start the experiment on Apr. 28 2015, we prepared the medicine and the equipment needed for the experiment before 10:00 am. Since then the mice were weighed and injected every day at 10:00 am;

3. Experimental arrangements:

| Day 1 | Day 3 | Day 5 |
|---|---|---|
| At 10:00 am of injection | At 10:00 am of | At 10:00 am of injection |
| after 60 minutes, the tail | injection after 60 | after 60 minutes, the |
| suspension test test was | minutes, the open field | forced swimming test |
| performed | test was performed | was performed |

4. Experimental environmental conditions
(1) Tail suspension test conditions: room temperature: 24° C., humidity: 62-67%.
(2) Forced swimming test conditions: room temperature:24° C., water temperature: 24° C., humidity: 62-67%, each two groups of experiments replace the water once.

B. The EXPERIMENTAL RESULTS AND ANALYSIS:

(1) Experimental results:

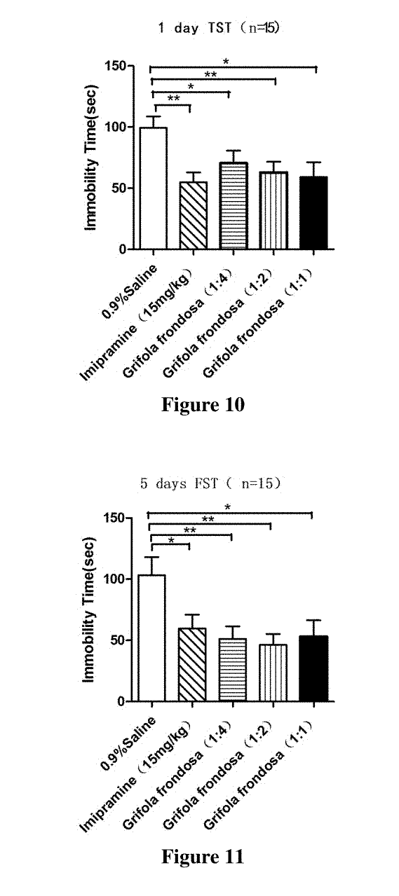

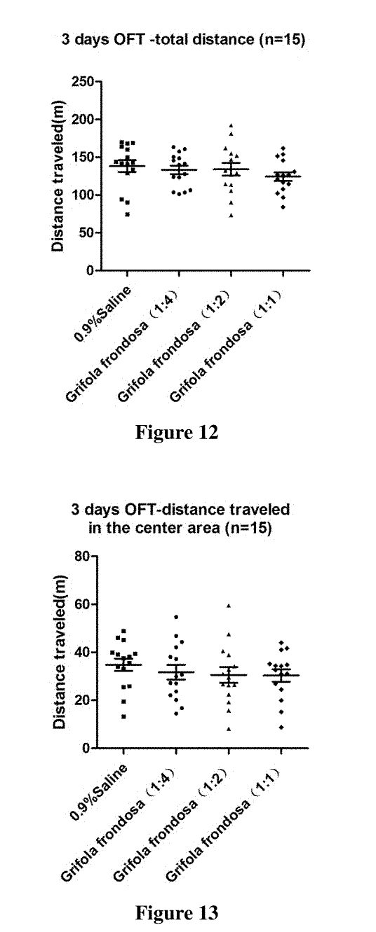

Behavioral results after different doses of Grifola frondosa treatment to CD-1 mice:
1) The results of tail suspension test after treatment with Grifola frondosa (15 repeat) for one day were shown in FIG. 10.
2) The results of forced swimming test after treatment with Grifola frondosa (15 repeat) for five days were shown in FIG. 11.
3) The results of the open field test after treatment with Grifola frondosa (15 repeat) for three days (total area moving distance and middle area moving distance) were shown in FIG. 12 and FIG. 13.

(2) Analysis of experimental results:

Analysis of the experimental results from the tail suspension test: the tail suspension test results of histogram after 1 day of Grifola frondosa (15 repeat) treatment were show in FIG. 10. It can be seen that Grifola frondosa (1:4) showed significant antidepressant effect (P=0.0449, P<0.01), Grifola frondosa (1:2) showed significant antidepressant effect (P=0.0075, P<0.001), and Grifola frondosa (1:1) showed significant antidepressant effect (P=0.0128, P<0.05). (As shown in FIG. 10)

Analysis of experimental results in the forced swimming test: the results of histogram of the forced swimming test after 5 days of Grifola frondosa (15 repeat) were presented in FIG. 11. It can be seen that Grifola frondosa (1:4) showed significant antidepressant effect (P=0.0079, P<0.01), Grifola frondosa (1:2) showed significant antidepressant effect (P=0.0030, P<0.001), and Grifola frondosa (1:1) showed significant antidepressant effect (P=0.0186, P<0.05). (As shown in FIG. 11)

Analysis of the Results in Open Field Test:

(1) The results of the distance traveled in the total area from the open field test after 3 days of Grifola frondosa (15 repeat) treatment were presented in FIG. 12. It can be seen that Grifola frondosa (1:4), (1:2), (1:1) showed no significant effect (P>0.05), and the results implied that the shortening of the immobilized time of the mice is not caused by the central excitatory effect of the medicament (Grifola frondosa). (As shown in FIG. 12)

(2) The results of the distance traveled in the center area of open field test after 3 days of the Grifola frondosa (15 repeat) treatment was presented in FIG. 13. It can be seen that Grifola frondosa (1:4), (1:2), (1:1) showed no significant effect (P>0.05), and the results further implied that the shortening of the immobilized time of the mice is not caused by the central excitatory effect of the medicament (Grifola frondosa). (As shown in FIG. 13)

1. An application of Maitake in the preparation of antidepressant medicaments is characterized as that the medicinal component of the antidepressant is selected from group of consisting of Maitake whole herb, Maitake extract, Maitake Polysaccharides D fraction, or Maitake Polysaccharides D fraction salts, esters or the glycosides synthesized by Maitake Polysaccharides D fraction and polysaccharides, wherein
said Maitake extract refers to product from following process: marinating the powder of the Maitake fruit body in pure hot water of 98° C. for 3 hours, the quality ratio of material to water is 1:30, pH 6.5˜7.0, and then concentrating the solution.

2. The application according to claim 1, the mentioned antidepressant medicament refers to a medicament that used for preventing or treating depression.

3. The use according to claim 1, the mentioned medicament is in dosage form of powder, table, capsule, solution, suspension, injecta or drip.

4. A kind of antidepressant medicament, it is characterized in: the medicinal component is selected from group of consisting of Maitake whole herb, Maitake extract, Maitake Polysaccharides D fraction, Maitake Polysaccharides D fraction salts, esters or the glycosides synthesized by Maitake Polysaccharides D fraction and polysaccharides, wherein said Maitake extract refers to product from following process: marinating the powder of the Maitake fruit body in pure hot water of 98° C. for 3 hours, the quality ratio of material to water is 1:30, pH 6.5˜7.0, and then concentrating the solution.

5. The medicament according to claim 4, the mentioned antidepressant refers to preventing or treating depression.

6. The medicament according to claim 5, the medicament is in the dosage form of powder, table, capsule, solution, suspension, injecta or drip.

7. The medicament according to claim 4, the mentioned medicament further contains pharmaceutically acceptable adjuvant.

8. The medicament according to claim 4, the mentioned medicament further contains pharmaceutical ingredient that plays a positive role in treating depression together with Maitake whole herb, Maitake extract, or Maitake Polysaccharides D fraction, or application a component that enhances the stability of Maitake whole herb, Maitake extract, or Maitake Polysaccharides D fraction.

9. An antidepressant method which characterized in that the treatment comprises providing an effective doses of medicament to subject on time, wherein medicinal component of mentioned medicament is selected from group of consisting of the Maitake whole herb, Maitake extract, or Maitake Polysaccharides D fraction, wherein said Maitake extract refers to product from following process: marinating the powder of the Maitake fruit body in pure hot water of 98° C. for 3 hours, quality ratio of material to water is 1: 30, pH 6.5˜7.0, and then concentrating the solution.

10. The method according to claim 9, said providing an effective doses of medicament to subject on time refers to providing Maitake Polysaccharides D fraction or Maitake extract with a dose of from 0.5 mg˜1000 mg/kg of body weight per day, or providing the Maitake whole herb together with food by a quality ratio of 1:1-4 between the Maitake whole herb and food.

11. The method according to claim 9, said providing effective doses of medicament to subject on time refers to providing Maitake Polysaccharides D fraction or Maitake extract by 5-15 mg/kg body weight per day.

12. The method of claim 9, the method of administration is oral, infusion or injection; the subject is a mammal, and the mentioned mammal comprising human.
